(19) United States
(12) Patent Application Publication
(10) Pub. No.: US 2023/0143233 A1
(43) Pub. Date: May 11, 2023

(54) CLOSED-LOOP SIMULTANEOUS FUNCTIONAL MAGNETIC RESONANCE IMAGING (fMRI)-ELECTROENCEPHALOGRAM (EEG)-TRANSCRANIAL MAGNETIC STIMULATION (TMS) SYSTEM

(71) Applicants: THE TRUSTEES OF COLUMBIA UNIVERSITY IN THE CITY OF NEW YORK, New York, NY (US); MEDICAL UNIVERSITY OF SOUTH CAROLINA, Charleston, SC (US)

(72) Inventors: Paul Sajda, New York, NY (US); Truman R. Brown, Mt. Pleasant, SC (US); Mark S. George, Sullivans Island, SC (US); Robin Goldman, Madison, WI (US); Josef Faller, Afing (AT); Jaycee Doose, Charleston, SC (US); James McIntosh, New York, NY (US); Joshua B. Teves, Charleston, SC (US); Yida Lin, Seattle, WA (US); Golbarg T. Saber, Chicago, IL (US); Aidan Blankenship, Charleston, SC (US); Spiro P. Pantazatos, New York, NY (US); Xiaoxiao Sun, Chongqing (CN)

(73) Assignees: THE TRUSTEES OF COLUMBIA UNIVERSITY IN THE CITY OF NEW YORK, New York, NY (US); MEDICAL UNIVERSITY OF SOUTH CAROLINA, Charleston, SC (US)

(21) Appl. No.: 18/054,447

(22) Filed: Nov. 10, 2022

Related U.S. Application Data
(60) Provisional application No. 63/277,860, filed on Nov. 10, 2021.

(51) Int. Cl. A61B 5/055; A61N 2/00; A61N 2/02; A61B 5/00

(52) U.S. Cl. CPC A61B 5/055; A61N 2/006; A61N 2/02; A61B 5/4064

(57) ABSTRACT

The present subject matter relates to techniques for systems and methods for determining alpha phase in brain of subjects undergoing depressive disorder. The disclosed system for a closed-loop operation in simultaneous functional magnetic resonance imaging (fMRI)-electroencephalogram (EEG)-transcranial magnetic stimulation (TMS), can include a processor that be configured to receive and process a functional magnetic resonance imaging (fMRI) data and/or an extracranial electroencephalogram (EEG) data and/or transcranial magnetic stimulation (TMS) pulse simultaneously.

### CROSS-REFERENCE TO RELATED APPLICATION

This application claims priority to U.S. Provisional Patent Application No. 63/277,860, which was filed on Nov. 10, 2021, the entire contents of which are incorporated by reference herein.

### GRANT INFORMATION

This invention was made with government support under grants MH106775 awarded by the National Institute of Health and N00014-20-1-2027 awarded by the Office of Naval Research. The government has certain rights in the invention.

### TECHNICAL FIELD

This present disclosure relates to a closed-loop system for treating depressive disorder, and more specifically, to a simultaneous functional magnetic resonance imaging (fMRI)-electroencephalogram (EEG)-transcranial magnetic stimulation (TMS) system.

### BACKGROUND

This Functional magnetic resonance imaging (fMRI) is a neuroimaging modality that can be used for cognitive neuroscience and clinical psychiatry. While fMRI scans can provide full brain coverage at a relatively high spatial resolution, temporal resolution can be limited due to a sluggish hemodynamic response.

Electroencephalograph (EEG) is a neuroimaging modality that can have high temporal resolution low spatial resolution. The cost of certain EEG scans can be lower than certain fMRI scans, e.g., less than $10 per scan with certain EEG equipment available for less than $50 thousand.

Transcranial magnetic stimulation (TMS) can treat depression, obsessive-compulsive disorder, or help smoking cessation. Additionally, TMS at a specific pulse range over the left dorsolateral prefrontal cortex (DLPFC) can be a treatment for pharmacologically resistant major depressive disorder (MDD).

There is a need for a technique to developing a combined system which can improve the timing the delivery of TMS relative to an endogenous brain state, e.g., to affect efficacy and short-term brain activity.

### SUMMARY

The disclosed subject matter provides techniques for systems and methods of closed-loop simultaneous functional magnetic resonance imaging (fMRI)-electroencephalogram (EEG)-transcranial magnetic stimulation (TMS).

In certain embodiments, the disclosed subject matter provides systems for identifying an alpha phase in brain of a subject. In some embodiments, the system includes a processor configured to process functional magnetic resonance imaging (fMRI) data and/or electroencephalogram (EEG) data, where the fMRI data and EEG data are simultaneously acquired, trigger a transcranial magnetic stimulation (TMS) pulse, analyze the fMRI data, the EEG data and the TMS pulse, and determine the alpha phase, wherein the alpha phase evokes strong activity in dorsal anterior cingulate cortex (dACC).

In certain embodiments, the processor further is configured to determine a target alpha phase associated with the strong response in the dACC as measured by blood oxygen level dependent (BOLD) signal. In certain embodiments, the EEG data is acquired using a MR-compatible EEG system.

In certain embodiments, a level of activation of the anterior cingulate cortex (ACC) varies with the TMS pulse applied to dorsal lateral prefrontal cortex (DLPFC). In certain embodiments, the DLPFC is depended on timing of the applied TMS pulses relative to the phase of an EEG alpha rhythm of the subject.

In certain embodiments, the alpha phase is determined in the EEG data at the time of each TMS pulse. In certain embodiments, the TMS pulse is synchronized to the patient's prefrontal quasi-alpha rhythm.

In certain embodiments, the disclosed subject matter provide a closed-loop electroencephalogram (EEG)-repetitive transcranial magnetic stimulation (rTMS) system. In one example, the system includes a processor configured to acquire EEG data, trigger a rTMS pulse; process the EEG data and the rTMS pulse; and determine a target alpha phase, where a first alpha phase is determined before the target alpha phase is determined.

In certain embodiments, rTMS is synchronized to the EEG alpha phase. In certain embodiments, the first alpha phase is determined by functional magnetic resonance imaging (fMRI) data, electroencephalogram (EEG) data, and a trigger transcranial magnetic stimulation (TMS) pulse.

In certain embodiments, the disclosed subject matter provides methods of determining a target alpha phase in brain of a subject. An example method includes performing a first simultaneous scan to determine a first alpha phase which evoked a strongest activity in a dorsal cingulate cortex (dACC), performing a treatment of multiple sessions to the subject, and performing a second simultaneous scan after the subject's treatment to determine a target alpha phase, wherein the simultaneous scan uses functional magnetic resonance imaging (fMRI) data, electroencephalogram (EEG) data, and a trigger transcranial magnetic stimulation (TMS) pulse.

In certain embodiments, a simultaneous EEG is recorded. In certain embodiments, a motor threshold is measured on the subject's left motor cortex.

In certain embodiments, the method further comprising adjusting a TMS output voltage until an involuntary thumb twitch is observed in the patient before the scan.

The disclosed subject matter provides a method for treating depressive disorder, comprising delivering EEG-triggered repetitive transcranial magnetic stimulation (rTMS) to a subject, wherein the rTMS is synchronized to the subject's prefrontal EEG quasi-alpha rhythm.

In certain embodiments, the rTMS is applied on the dorsal prefrontal cortex (DLPFC).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A provides a flowchart of an example closed-loop stimulation system in accordance with the disclosed subject matter. FIG. 1B provides a diagram showing example logic of the closed-loop stimulation system that synchronized the onset of rTMS to EEG alpha phase.

FIG. 2 provides a diagram showing an example different operation mode switch for the system in FIG. 1B.

FIG. 3A provides a diagram showing example results of modelling BOLD response as a sinusoid relative to the alpha oscillation. FIG. 3B provides a diagram showing example results of modelling BOLD response as a sinusoid relative to the alpha oscillation raw data (dots) overlaid. FIG. 3C provides a diagram showing a Markov Chain Monte Carlo (MCMC) result.

FIG. 4 provides a diagram showing example pre-treatment and post-treatment design.

FIG. 5 provides a diagram showing an example trial-weighted inter-trial phase coherence (ITPC) calculation.

FIG. 6A provides a diagram showing estimation of phase entrainment in the quasi-alpha band (6-13 Hz) at the target electrode (F3) from SYNC subject group; FIG. 6B provides a diagram showing estimation of phase entrainment in the quasi-alpha band) 6-13 Hz) at the target electrode (F3) from UNSYNC subject group.

FIG. 7A provides a diagram showing an example change in quasi-alpha entrainment between a first and last session. FIG. 7B provides a diagram showing example change in quasi-alpha entrainment between the first and last session, wherein pre- and post-treatment ITPC^{max} values are not derived from single sessions. FIG. 7C provides a diagram shows the difference (Δφ∈[0, π]) between the preferred phases (φₚᵣₑ and φₚₒₛₜ) and the phases at which ITPC peaked post-rTMS, for an example SYNC group; FIG. 7D provides a diagram shows the difference (Δφ∈[0, π]) between the preferred phases (φₚᵣₑ and φₚₒₛₜ) and the phases at which ITPC peaked post-rTMS, for an example UNSYNC group.

FIG. 8A provides a diagram showing a model prediction of changes in ITPC^{max[1]} between the first and last session for SYNC and UNSYNC groups at the ROI near the rTMS target ROI; FIG. 8B provides a diagram showing a corresponding model contralateral to the target ROI, FIG. 8C provides a diagram showing a corresponding model in the medial-frontal ROI and FIG. 8D provides a diagram showing a corresponding model in the occipital ROI.

It is to be understood that both the foregoing general description and the following detailed description are exemplary and are intended to provide further explanation of the disclosed subject matter.

### DETAILED DESCRIPTION

The disclosed subject matter provides techniques for identifying or determining alpha phase in brain of patients who suffer from depressive disorder, through acquiring and analyzing data, stimulation, and subsequent application of a guided and modified stimulation to patients. The disclosed subject matter includes systems and methods of a closed-loop simultaneous functional magnetic resonance imaging (fMRI)-electroencephalogram (EEG)-transcranial magnetic stimulation (TMS).

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

The term “coupled,” as used herein, refers to the connection of a device component to another device component by methods known in the art.

As used herein, “treatment” or “treating” refers to inhibiting the progression of a disease or disorder, or delaying the onset of a disease or disorder, whether physically, e.g., stabilization of a discernible symptom, physiologically, e.g., stabilization of a physical parameter, or both. As used herein, the terms “treatment,” “treating,” and the like, refer to obtaining a desired pharmacologic and/or physiologic effect. The effect can be prophylactic in terms of completely or partially preventing a disease or condition or a symptom thereof and/or can be therapeutic in terms of a partial or complete cure for a disease or disorder and/or adverse effect attributable to the disease or disorder. “Treatment,” as used herein, covers any treatment of a disease or disorder in an animal or mammal, such as a human, and includes: decreasing the risk of death due to the disease; preventing the disease or disorder from occurring in a subject which can be predisposed to the disease but has not yet been diagnosed as having it; inhibiting the disease or disorder, i.e., arresting its development (e.g., reducing the rate of disease progression); and relieving the disease, i.e., causing regression of the disease.

As used herein, the term “subject” includes any human or nonhuman animal. The term “nonhuman animal” includes, but is not limited to, all vertebrates, e.g., mammals and non-mammals, such as nonhuman primates, dogs, cats, sheep, horses, cows, chickens, amphibians, reptiles, etc. In certain embodiments, the subject is a pediatric patient. In certain embodiments, the subject is an adult patient.

As used herein, the term “MDD” refer to major depressive disorder of a subject. In certain embodiments, for those patients who are suffering or have suffered from MDD, they underwent repetitive transcranial magnetic stimulation (rTMS) or other suitable therapy as a treatment.

As used herein, the term “fET” refer to a simultaneous system combining functional magnetic resonance imaging (fMRI)-electroencephalogram (EEG)-transcranial magnetic stimulation (TMS), either referring to a system or process, e.g., fMRI-EEG-TMS. In certain embodiments, fET can be used to determine alpha phase of patients.

As used herein, the term “BOLD” refers to signal or response of blood oxygen level dependent in a measurement of brain activation. In certain embodiments, BOLD response can be evoked by TMS or other suitable stimulation.

As used herein, the term “ACC” refers to anterior cingulate cortex of brain. In certain embodiments, the level of activation of ACC is measured to determine the alpha phase. In certain embodiments, some subject-specific preferred alpha phase which evoked strongest activity in dorsal anterior cingulate cortex (dACC) is determined by using a simultaneous fET.

As used herein, the term “DLPFC” refers to left dorsolateral prefrontal cortex.

As used herein, the term “SYNC” refers to a synchronization to alpha or quasi-alpha activity, and the term “UNSYNC” refers to a nonsynchronization to alpha or quasi-alpha activity.

As used herein, the term “alpha phase” refers to a type of brain waves, which is produced when brain is relaxed or not stressful. In certain embodiments, a prefrontal alpha phase φₚᵣₑ (or φₚᵣₑ) is the phase which yielded the strongest BOLD fMRI activation in the ACC. In certain embodiments, a target alpha φ_{targ} (or φ_{targ}) phase refers to an optimized phase.

As used herein, the term “activity” refers to brain activity. Alpha phases or waves substantially affect brain activity. Strong activity promotes the production of neurotransmitters, which is beneficial for reducing symptoms of anxiety and depression.

As used herein, the terms “Pre” and “Post” are two segmented and separate datasets from EEG data. For dataset Pre, epochs are extracted from the intervals between two rTMS pulse trains. For dataset Post, epochs were extracted from a time window, e.g. [0, 2.5] s relative the last pulse of each pulse train.

The disclosed subject matter provides techniques for simultaneous functional magnetic resonance imaging (fMRI)-electroencephalogram (EEG)-transcranial magnetic stimulation (TMS). An example fMRI-EEG-TMS (fET) system can identify a specific phase of each individual patient's alpha rhythm to improve or optimize the neural response of the anterior cingulate cortex (ACC) following a TMS pulse to the dorsolateral prefrontal cortex (DLPFC). In certain embodiments, the selected individualized phase can be used as the phase at which repetitive transcranial magnetic stimulation (rTMS) can be triggered via a closed-loop EEG-rTMS (no fMRI) instrument that can perform real-time analysis of the patient's EEG to deliver the first pulse of rTMS during treatment.

In certain embodiments, the disclosed system can be configured to simultaneously perform fMRI, EEG, and TMS. For example, the disclosed system can include a 43 channel MR compatible bipolar EEG system, an MRI Scanner (e.g., with a 12 channel head coil), and a TMS neurostimulator (e.g., with a TMS coil positioner and MRI compatible setup). In non-limiting embodiments, a motor threshold can be measured on the subject's left motor cortex, adjusting the TMS output voltages until an involuntary thumb twitch is observed before the scan.

In certain embodiments, the TMS coil can be configured to a subject-specific intensity (e.g., about 100 to 120% of the motor threshold). In non-limiting embodiments, functional imaging can be performed with a 12-channel head coil and a multi-echo multiband pulse sequence. A high-resolution structural T1 image can be acquired for registration as well as a field map for distortion correction using the 12 channel head coil. In non-limiting embodiments, the high resolution structural T1 image can be collected using, e.g., a 32 channel head coil in a separate session prior to a second session for the 3-way fMRI-EEG-TMS (fET) acquisition.

In certain embodiments, sequence parameters can be modified. For example, sequence parameters can be 3.2×3.2×3.2 mm voxel size, 36 slices, MB acceleration factor 2, repetition time (TR) 1600 ms, echo time 1 (TE1) 11 ms, TE2 32.16 ms, TE3 53.32 ms. In non-limiting embodiments, the inter-pulse interval can be pseudorandomized and range from four to six TRs. Four to six runs were collected for each subject yielding 184 to 276 TMS pulses per session. In non-limiting embodiments, sequence parameters in the replication dataset can be multiband acceleration factor=2, TE1=11.20 ms, TE2=32.36 ms, and TE3=53.52 ms. Whole-brain fMRI can be acquired (e.g., in 38 slices, voxel size 3.2×3.2×3.2 mm, TR 1.75 s, flip angle 58 degrees, with a 200 ms TR gap). Reverse phase encode sequences can be acquired to unwarp the images.

In certain embodiments, the MR compatible bipolar EEG cap (e.g., with 36 electrodes) can be placed on the subject's head. Individual impedances can be reduced with electrode gel to below 20 k. The location of the DLPFC can be measured and marked (under the F3 electrode) for TMS coil placement in the MRI scanner. The TMS coil can run through a filter into the MRI room and be placed over the subject's DLPFC outside the EEG cap. The EEG cap can be connected to an MRI-compatible amplifier that can sample at 488 Hz with a gain of 400. A 12 channel head coil can be used to acquire both the anatomical and BOLD scans. In non-limiting embodiments, EEG can be simultaneously acquired at a 488 Hz sampling rate. In non-limiting embodiments, all system clocks can be synchronized with the 10 MHz scanner clock. The scanner trigger can initiate the EEG acquisition for each run. TMS pulse firing and timing can be automatically controlled via a suitable program synchronized with the scanner trigger.

In certain embodiments, each TMS pulse can be fired at the beginning of a 200 ms gap at the end of each TR. The gap can allow the magnetic effects of the TMS pulse to not affect subsequent image acquisition. In non-limiting embodiments, EEG can also be simultaneously acquired at a 488 Hz sampling rate. Subjects can be instructed to keep their eyes open and look at a fixation cross during all runs.

In certain embodiments, the disclosed subject matter provides an EEG-rTMS system. For example, the head circumference can be used to select an appropriately sized cap with 32 active EEG sensors, which can be placed on the patient's head. Cap placement can be verified by making sure the EEG sensor for a channel (Cz) is located midway between nasion and inion as well as between the left and right preauricular points. In non-limiting embodiments, impedance can be reduced to less than 10 kΩ for each electrode. EEG can be sampled at 10 kHz using a biosignal amplifier. This amplifier can be designed to recover from electromagnetic artifacts related to a TMS pulse in less than 1 ms. In non-limiting embodiments, additional high-pass filters can be omitted before recording the data. In certain embodiments, synchronized acquisition of all signals and experimental events can be accomplished through the software framework Labstreaming Layer (LSL), and all data can be stored in extensible data format (XDF) files. In certain embodiments, the processor administers rTMS are locked to the phase of the alpha wave in the EEG.

In certain embodiments, the resting motor threshold (MT) can be measured at the resting state of the beginning of the first treatment session. MT can be acquired using single-pulse TMS, which provides a noninvasive index of cortical excitability. In non-limiting embodiments, sequential testing (PEST) can be used for threshold estimation. For example, motor response to TMS can be defined as any finger movement in the right hand. For PEST determination, a pulse can be delivered at a certain intensity and then the experimenter can report whether or not they observed a motor response. This process can be repeated in subsequent trials until the PEST algorithm converged to a precise resting MT estimate.

During treatment, TMS intensity can be gradually configured from measured subject-specific MT up to 120% of this MT (e.g., 1st 100% MT, 2nd 110% MT, and 3rd+ 120% MT). The scalp location over the left DLPFC, which can be targeted with rTMS, can be determined by taking measurements with the EEG cap on. Nasion to inion, tragus to tragus, and head circumference can be measured and added into the Beam F3 software as inputs. This can provide two coordinates, distance along circumference from the midline (X) and distance from vertex (Y), which can be used to identify the location of F3, which can be then marked, e.g., on a personalized swim cap for reference for future treatment sessions.

In certain embodiments, every treatment session can start with a “resting state” recording of five minutes of EEG, for which patients can be instructed to keep their eyes open and visually fixate a point marked by a grey cross (e.g., 18 cm wide, 18 cm tall, 2 cm line thickness), 110 inches in front of them. This recording can be used to determine the individual alpha frequency (IAF), picked from a range between 6 and 13 Hz, as determined by baseline power in the alpha band. This recording can be further used to improve or optimize individual thresholds for triggering TMS, represented by a model fit parameter called Root Mean Square Error (RMSE). This optimization can be performed to ensure the efficiency of the system (e.g., the system did not take longer than approximately 5 seconds to identify an appropriate EEG target because 3000 TMS pulses in one treatment session can be delivered in no more than approximately 30 minutes). An identical five-minute resting state recording can be collected at the end of each treatment session.

In certain embodiments, the disclosed system can administer rTMS locked to the phase of the alpha wave in the EEG. For example, the disclosed system can read densely sampled (10 kHz) EEG from the amplifier in chunks of 20 samples. After applying a finite impulse response (FIR)-based antialiasing filter with a cut-off frequency at 50 Hz, the EEG data can be downsampled to 500 Hz by retaining only every 20th sample. The left lateralized prefrontal alpha oscillation can be recovered by first spatially averaging the signal for the EEG channels F3, F7 and FP1, and by applying a causal FIR-based band-pass filter with corner frequencies at IAF±2 Hz. The disclosed system then can fit a model based on a single sinusoid to the filtered signal in the time window [−300, −100] ms using ordinary least squares regression. This can allow for flexibility in terms of the exact frequency by first fitting the model for multiple frequencies.

In non-limiting embodiments, candidate frequencies can be fc where {fc∈R|fc=IAF−3+ξ++0.5*k and fc<IAF+3} for k=0 . . . 12 and where can be drawn randomly from a uniform distribution over the range 0 to 0.5. The frequency in fc that can be associated with the lowest RMSE fit can be used to predict the signal for a test window [−100 0] ms. When the RMSE between prediction and signal in the test window is under a specific subject-specific threshold, the time point for the next peak in the alpha rhythm can be predicted and scheduled for the future triggering of an rTMS pulse train. In non-limiting embodiments, the logic can continue to preprocess data, but no new model fitting attempts can be started until the scheduled time-point is reached. Once the scheduled time point is reached, an rTMS pulse train can be triggered. Triggering can be followed by a refractory period (e.g., of 3 s), in which new EEG data can be preprocessed but sine fitting and triggering remained disabled.

In certain embodiments, the disclosed system can be operated in a closed-loop mode. Unlike an open-loop EEG-rTMS system, the closed-loop operation in the EEG-rTMS system can provide real-time EEG artifact correction and new online machine learning algorithms for improved flexibility in the type of EEG signatures that can be used to control the timing of the TMS triggering.

In certain embodiments, instead of using a single TMS pulse, the disclosed system can use repetitive TMS, which can include multiple TMS pulses for the TMS session. In non-limiting embodiments, the disclosed system can achieve phase-locked triggering at any target phase with high accuracy.

In certain embodiments, the disclosed system can include three modalities (e.g., MRI, EEG, and TMS) together and can achieve simultaneous data recording of fMRI and EEG. The disclosed subject matter also provides a data processing pipeline for the calculation of individual phases associated with maximum BOLD activation in the dorsal anterior cingulate cortex (ACC) region. The EEG-TMS system can also perform phase-locked TMS pulse triggering with high accuracy at any target phase. In some embodiments, the close-loop fET can combine the function of open-loop fET and close-loop EEG-TMS to achieve the real-time data analysis and phase-locked TMS pulse triggering. In non-limiting embodiments, the disclosed system can be used directly for data generated by another group.

In certain embodiments, the disclosed system can be an fET instrument, with both open and closed-loop capabilities that can be made widely available and that permit precise triggering on a range of brain states. These capabilities can allow researchers and clinicians to address a broad set of research and clinical needs as well as a large range of experimental parameters, which affect brain stimulation following TMS. In non-limiting embodiments, the disclosed system can be used as an efficient tool for treating major depression disorder and improving neuroplasticity.

### EXAMPLE

### Example 1: The Logic of the Closed-Loop Stimulation System that Synchronizes the Onset of rTMS to EEG Alpha Phase

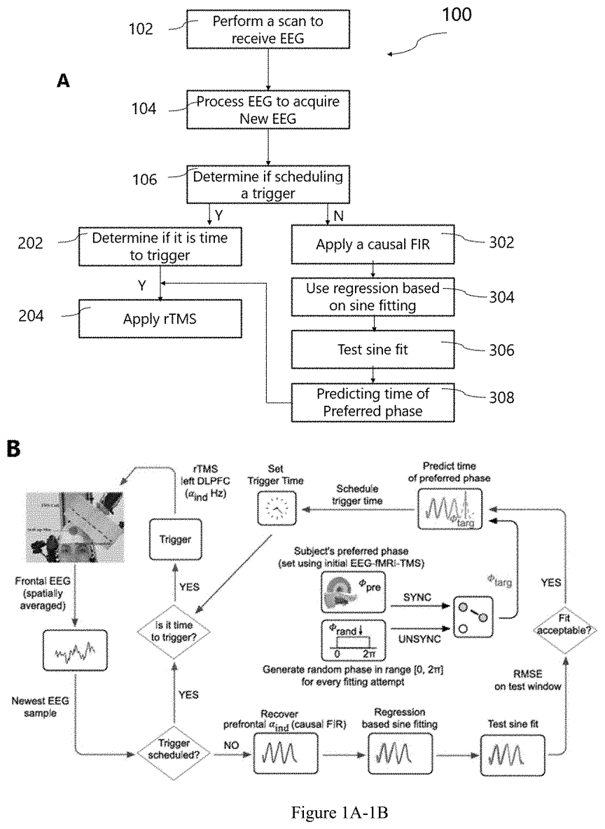

In this example, a closed-loop neurostimulation system illustrated in FIG. 1A-1B was used to show that synchronized application across weeks of rTMS treatment can yield increased entrainment.

This example investigated whether, for a multi-week daily treatment of repetitive TMS (rTMS), there is an effect on brain activity that depends on the timing of the TMS relative to individuals' prefrontal EEG quasi-alpha rhythm (e.g., between 3 and 13 Hz).

A closed-loop system that delivers specific EEG-triggered rTMS to patients undergoing treatment for major depressive disorder (e.g., MDD) was used. In a double blind study, patients received daily treatments of rTMs over a period of several weeks (e.g., 6 weeks) and were randomly assigned to either a synchronized or unsynchronized treatment group, where synchronization of rTMs was to their prefrontal EGG quasi-alpha rhythm.

FIG. 1A, provides a flowchart 100 of an example closed-loop system. At 102, a scan on the subject's brain is performed to receive EEG. At 104, EEG information is processed to acquire a new EGG sample. At 106, a determination of whether a trigger is scheduled. If the result of 106 is YES (Scheduled), then proceeding with 202, a determination is made whether it is time to trigger. At 204, upon YES in 202, rTMS is applied on to subject's brain.

If the result of 106 is NO (not Scheduled), then proceeding with 302, a causal finite impulse response (FIR) is applied to recover left lateralized prefrontal alpha oscillation. At 304, an ordinary least squares regression is applied based on sine fitting to fit a model. At 306, a test is made to determine whether the sine fit is acceptable. If the result of 306 is YES, proceeding with 308, the time point for the target phase is predicted, and a trigger time of an rTMS pulse is scheduled. Following to 204, a trigger of rTMS is performed upon the scheduled time point to the brain of the subject. In certain embodiments, the φ_{targ} phase of a subject in SYNC can be determined in fET system. In certain embodiments, the φ_{targ} phase of a subject in UNSYNC can be determined in an generation of random phase in range [0, 2π] for everything fitting attempt.

FIG. 1B shows example logic of the closed-loop for identifying a specific phase of each individual patient's alpha rhythm to improve or optimize the neural response of the anteri-or cingulate cortex (ACC) following a TMS pulse to the dorsolateral prefrontal cortex (DLPFC). An example system can include a processor. The processor can be configured to perform the instructions specified by software stored in a hard drive, a removable storage medium, or any other storage media. The software can be written in a variety of languages, e.g., MATLAB and/or Microsoft Visual C++. Additionally or alternatively, the processor can include hardware logic, such as logic implemented in an application-specific integrated circuit (ASIC). The processor can be configured to control one or more of the system components.

The software read densely sampled (10 kHz) EEG from the amplier can be set, e.g., in chunks of 20 samples. After applying a causal finite impulse response (FIR)-based antialiasing filter with a cut-off frequency at 50 Hz, the EEG data was downsampled to 500 Hz by retaining only every 20th sample. Next, the left lateralized prefrontal alpha oscillation was recovered by first spatially averaging the signal for the EEG channels F3, F7 and FP1 and by applying a causal finite impulse response (FIR) based band-pass filter with corner frequencies at IAF 2 Hz. The software then fit a model based on a single sinusoid (i.e., on the feature sin(2π*f*t) and cos(2π*f*t)) to the filtered signal in the time window [−300, ˜−100] ms using ordinary least squares regression. This allowed for flexibility in terms of the exact frequency by first fitting the model for multiple frequencies.

Candidate frequencies were f_{c} where {f_{c}∈R|f_{c}=IAF−3+ε+0.5×k and f_{c}<IAF+3} for k=0 . . . 12 and where ε was drawn randomly from a uniform distribution over the range 0 to 0.5 (ε˜(0, 0:5)). The frequency in f_{c} that was associated with the lowest RMSE fit was used to predict the signal for a test window [−100, 0] ms. The time point for the next peak in the IAF rhythm was predicted and scheduled for future triggering of an rTMS pulse train only if the RMSE between prediction and signal in the test window were under a specific subject specific threshold. The logic continued to pre-process data, but no new model tting attempts were started until the scheduled time-point was reached.

Once the scheduled time point was reached, an rTMS pulse train was triggered via parallel port output from the control computer to a microcontroller-based safety monitoring circuit, which ensured stimulation could not go above 14 Hz maximum or 3000 pulses total for a given treatment session. Each of the 40 pulses in a pulse train was individually triggered with a delay between pulses of 1/IAF. Triggering was always followed by a refractory period of 2*40* (1/IAF), so that the OFF time following stimulation was at minimum 2 times the length of the ON time for stimulation (see FIG. 1A-1B). During this refractory period, new EEG data was preprocessed but sine tting and hence triggering remained disabled.

The phase at which rTMS was synchronized the first pulse in each TMS pulse train is based on a unique targeting approach using an intergarted fET system. In the example, it was determined whether rTMS applied synchronized or unsynchronized to this phase over 30 sessions of treatment impacts entrainment over time.

TABLE 1

| | # of | | | Duration of Current |
|---|---|---|---|---|
| | Patients | Age (y) | Sex | Depressive Episode |
| SYNC | 7 | 50.1 ± 10.5 | 7 6 F, 1 M | 50.1 ± 39.9 weeks |
| UNSYNC | 8 | 8 45.0 ± 15.9 | 6 F, 2 M | 60.6 ± 60.5 weeks |
| Total | 15 | 47.4 ± 13.4 | 12 F, 3 M | 55.7 ± 50.4 weeks |

Number of patients in every group, average age, gender, and average (± standard deviation) duration of the current depressive episode in weeks are shown. The duration of the current depressive episode is used to describe how long an individual patient has been depressed during the present depressive episode. There is no significant difference between the SYNC and UNSYNC groups in age (p = 0.480 3) or duration of current depressive episode (p = 0.703 4).

In the example, an interim blinded analysis of an ongoing clinical trial was built. All EEG data for this randomized, double-blind, active comparator-controlled clinical trial was collected. 23 patients were consented and enrolled in the example, and 15 (see Table 1) were able to complete the rTMS treatment. Eight subjects dropped out for reasons including claustrophobia (N=2, i.e., could not complete MRI), hospital admission due to severe depressive episodes (N=1), and some participants could no longer make the time commitment for the study (N=5). During enrollment, all patients were randomly assigned to the SYNC or UNSYNC group before treatment.

The inclusion criteria included diagnosis of unipolar MDD in a current major depressive episode, Hamilton Rating Scale for Depression (HRSD) score 20, age between 21 and 70, and fixed and stable antidepressant medications for 3 weeks prior to and during the trial. Patients also needed to show a moderate level of resistance to antidepressant treatment, defined as failure of one to four adequate medication trials, or intolerance to at least three trials. Primary exclusion criteria were that patients had to be able to undergo a 3T MM scan as well as TMS treatment safely. To ensure that baseline level of depression severity was stable at the time of study enrollment, patients were dropped from the study if they showed more than 30% improvement in the HRSD score from the time of their initial screening to the baseline assessment.

The system of FIG. 1B illustrates the logic of the closed-loop system that synchronized the onset of rTMS to EEG alpha phase. The system of FIG. 1B continuously processed EEG in real-time, where the EEG was sampled at 10 kHz. To optimize throughput, data was read from the amplifier in chunks of 20 data points (i.e., samples). Subsequently, a low-pass antialiasing filter was applied with a cut-off at 50 Hz and the signal was downsampled to 500 Hz.

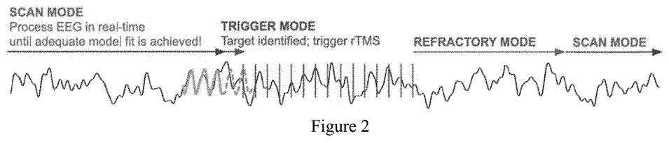

As illustrated in FIG. 2, for EEG processing, the logic switched between three operation modes, SCAN MODE (blue arrows), TRIGGER MODE (red arrows) and REFRACTORY MODE (grey arrow). Model fitting in SCAN MODE was performed in parallel to reading new data (multi-threading) and every new fitting attempt was always performed on the newest available data. Starting in SCAN MODE, the system fitted multiple single-sine function models on to the individual patient's prefrontal quasi-alpha signal (α_{ind}, 6-13 Hz; spatial average of FP1, F7 and F3) in a time window [−300, ˜−100] ms relative to the newest EEG sample. The resulting model that achieves the lowest root mean square error (RMSE) on that training signal was used for prediction on a more recent test signal in the time window [−100, 0] ms, again relative to the newest EEG sample.

If the RMSE on that test signal does not reach below a pre-determined, subject-specific threshold, the logic continued with a new fitting attempt, but now again using data relative to the newest EEG data that arrived in real-time. Otherwise, if and only if the RMSE on that test signal was below this threshold, the single-sine model was used to predict the prefrontal quasi-alpha wave up to 123 ms into the future. The targeted phase, φ_{targ}, then depended on the randomized treatment arm for that patient. For SYNC, φ_{targ} is the subject specific preferred phase, φₚᵣₑ that was determined in an initial combined fMRI-EEG-TMS system (fET). For UNSYNC, φ_{targ} was drawn from a uniform random distribution over the range [0, 2π] at every prediction (φ_{targ}˜U(0, 2π)).

Taking into account the group delay of causal filtering and processing time, the logic then scheduled the rTMS trigger onset at the predicted future time of φ_{targ} and switched into TRIGGER MODE. In TRIGGER MODE, no model fitting is attempted. Instead the logic keeps reading new data samples. Whenever the scheduled trigger time has arrived, a train of 40 TMS pulses is triggered where the inter-pulse-interval is the reciprocal of the subject's individual alpha frequency (IAF, Δₜᵢₚᵢ=1/IAF). Directly after the 40th pulse has been triggered, the logic switches into REFRACTORY MODE, where the system does nothing other than reading in new EEG samples for 2*4*1/IAF or twice the amount of time it took to deliver 40 TMS pulses, after which the logic again switches into SCAN MODE.

### Example 2: Determining Alpha Phase Using a Combined fET System

Each patient underwent a simultaneous fET scan at the beginning of the study to determine a subject-specific target alpha phase which evoked strongest activity in dorsal anterior cingulate cortex (dACC). The patients also underwent a second fET scan at the end of their treatment (after 30 sessions) to determine the target phase after the treatment was complete. Simultaneous EEG was recorded using a MR-compatible EEG system. A 3T Siemens Prisma MRI scanner (Siemens, Munich, Germany) was used to collect functional echo-planar image (EPI) data. A Rapid2 system (Magstim, Whitland, UK) was used for TMS pulse delivery and the timing of delivery was controlled via an E-Prime 2 (Psychology Software Tools, PA, USA) program synchronized with the scanner trigger. Stimulator intensity was set at 120% of the subject's motor threshold. Each TMS pulse was red at the beginning of a 200 ms gap at the end of each TR (i.e., repetition time in fMRI pulse sequence). In a subsequent analysis, the corresponding phase of frontal alpha was estimated in the EEG at the time of each TMS pulse. The target phase was then selected as the phase that was associated with the strongest response in the dACC as measured by the BOLD (blood oxygen level dependent) signal.

In this example, it was hypothesized that the level of activation of the anterior cingulate cortex (ACC) varies following a transcranial magnetic stimulation (TMS) pulse applied to the DLPFC, with a dependence on the precise timing of the applied TMS pulses relative to the phase of the individual subject's EEG alpha rhythm (in measurement, the EEG frequency range was expanded to 6 to 13 Hz for the alpha phase-locking in the EEG-rTMS system, and therefore refer to this rhythm as “quasi-alpha” EEG in the example). To increase the effect of the weeks of EEG-synchronized Repetitive TMS (rTMS) in this clinical trial, it was thus determined a “target phase” (i.e., the target phase in the EEG quasi-alpha cycle relative to which we triggered the first TMS pulse of a rTMS pulse train if this individual patient was assigned to the SYNC group) once initially when subjects were enrolled using a combined fET system. Specifically, EEG was used to estimate the instantaneous subject specific alpha phase in frontal regions covering the DLPFC prior to TMS pulse delivery, and measure activity in the ACC BOLD signal to assess target engagement. In a model of the example the phase as, a phase shift between the alpha rhythm and BOLD response as the symbol alpha α^{c}, and the BOLD response as y:

y=b₀^{c}+A·cos(α^{c}+⇔y=b₀^{c}+A cos α^{c} cos ϕ−A sin α^{c} sin ϕ⇔y=b₀^{c}+b₁^{c} cos ϕ+b₂^{c} sin ϕ (1)

Where b₀^{c}=A cos α^{c}, and b₂^{c}=−A sin α^{c}. Since sin² α+cos² α=1, it can be derived, based on tangent function, rewriting this formula in matrix form, the following is obtained:

$$Y = {XB}^{c} + ε \qquad ( 2 )$$

$$⇔ [ \begin{matrix}{y}_{0} \\ {y}_{1} \\ … \\ {y}_{n}\end{matrix} ] = [ \begin{matrix}{x}_{0}^{0} & {x}_{0}^{1} & {x}_{0}^{2} \\ {x}_{1}^{0} & {x}_{1}^{1} & {x}_{1}^{2} \\ … & … & … \\ {x}_{n}^{0} & {x}_{n}^{1} & {x}_{n}^{2}\end{matrix} ] \times [ \begin{matrix}{b}_{0}^{c} \\ {b}_{1}^{c} \\ {b}_{2}^{c}\end{matrix} ] + ε \qquad ( 3 )$$

where xᵢ⁰=1, xᵢ¹=cos ϕ and xᵢ²=sin ϕ.

Using the equations above, the BOLD response with a sinusoidal model of phase was explained. The maximum of the sinusoid from this model for the first fET scan, outputs a pre-treatment preferred phase (pre) for each subject, and it was used as the target phase (targ) of TMS pulse triggering in the EEG-rTMS treatment sessions for subjects in the SYNC group. This method is repeated using a Bayesian approach to generate estimates of how much of effectiveness of the estimates of coefficients mapping phase to the BOLD response.

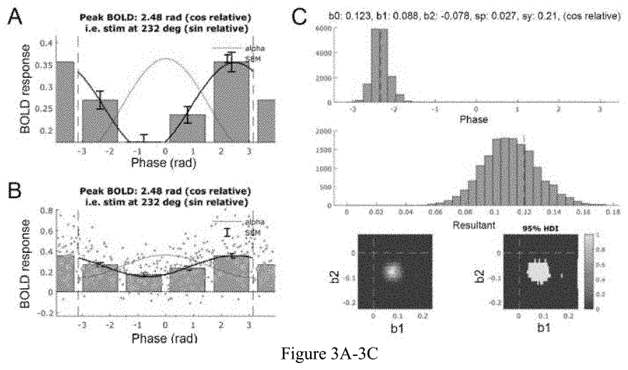

Referring to FIGS. 3A-3C, a report of the preferred phase calculation for each subject was generated. The target phase derived previously was used over the subsequent weeks of EEG-rTMS treatment if and only if this subject was assigned to SYNC group. FIG. 3A shows the result of modelling BOLD response as a sinusoid relative to the alpha oscillation. The histogram shows binned response with standard error of the mean (SEM). The black line represents the model fit while the red line shows the corresponding alpha oscillation which is defined entirely by the x-axis. In this example, peak BOLD is reached at 2.48 radrelative to a cosine which means 4.06 rad relative to a sine function.

FIG. 3B is the same as FIG. 3A, but with raw data (blue dots) overlaid. FIG. 3C includes a Markov Chain Monte Carlo (MCMC) result which provides an idea in parameter estimates. In the example, FIG. 3A-3C include one subject's model result of assumed model described in Equation (3), where b₀^{c}=0:254, b₁=0:062, b₂=0.0:080 relative to a cosine, so there was a target a peak at 2.48 rad relative to a cosine function (i.e., 4.06 rad relative to a sine function). Because all preferred phases of each subject are determined relative to a sine function, this subject should be stimulated at 232 degrees (ϕ=4:06 rad=4:06/π*180°=˜232° relative to a sine wave if they belong to the SYNC group (SYNC: ϕ_{targ}=ϕₚᵣₑ). For subjects that were assigned to the UNSYNC treatment group, a random value for targ was drawn and targeted for every first TMS pulse in each rTMS pulse-train (UNSYNC: ϕ_{targ}˜(0, 2π)). At the end of all treatment sessions, a second fET scan was done to measure the post-treatment preferred phase (φₚₒₛₜ) for each subject with the same estimation method described above.

### Example 3: A Closed-Loop EGG-rTMS System

A closed-loop EGG-rTMS system is developed in the example. Head circumference was used to select an appropriately sized cap with 32 active EEG sensors (ActiCap Slim, Brain Products GmbH, Munich, Germany), which was placed on the patient's head. Cap placement was verified by making sure the EEG sensor for channel Cz was located midway between nasion and inion as well as between the left and right preauricular points. Impedance was reduced to less than 10 kΩ for each electrode. EEG was sampled at 10 kHz using a biosignal amplifier (ActiChamp, Brain ProductsGmbH, Munich, Germany). This amplifier is designed to recover from electromagnetic artifacts related to a TMS pulse in less than 1 ms. No additional high-pass filters were applied before recording the data. Synchronized acquisition of all signals and experimental events was accomplished through the software framework Lab streaming Layer (LSL) and all data was stored in extensible data format (XDF) files.

Prior to EEG analysis, a double exponential model was fit to the average post-pulse response from t=17.5 ms to t=Δₜᵢₚᵢ, which is the interval between pulses in a train, i.e., 1/IAF. This fit was then subtracted from the post-pulse response for all pulses in a session in order to suppress a slow instantaneous TMS artifact present in the EEG. This instantaneous TMS artifact was interpolated from 1 ms to 17.5 ms. The entire EEG session was then low-pass filtered with a cut-off at 50 Hz and down-sampled to 250 Hz. Infomax-based Independent Component Analysis (ICA) was then performed on each session for each subject independently. The CORRMAP plugin for the EEGLAB MATLAB toolbox was used to identify ocular artifacts across sessions and those components were subsequently removed from the EEG data. For consistency with other studies in this project, data was then re-referenced to electrode location TP10 (close to the right mastoid). The arithmetic mean was computed separately for every EEG channel and subtracted from every point in the time series for that channel.

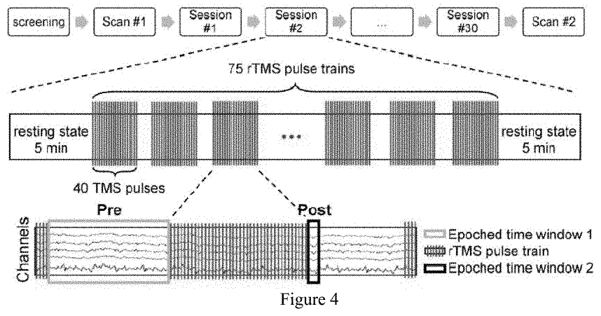

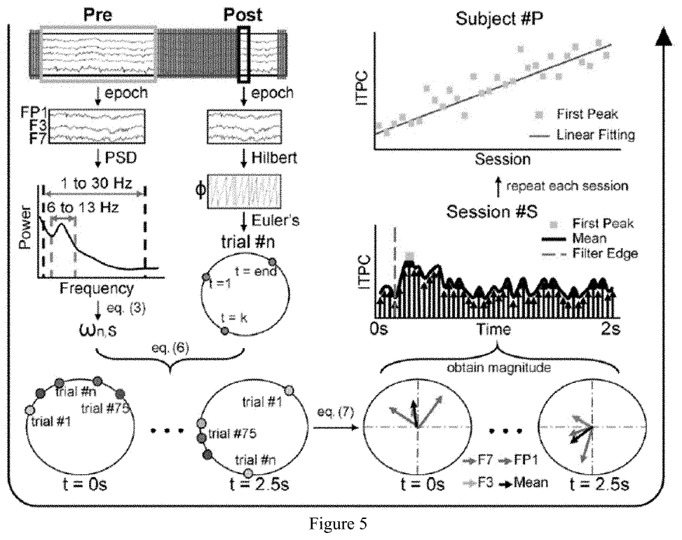

Next, EEG data was segmented into two separate datasets (Pre and Post) for two separate calculations (see FIG. 4 and FIG. 5). For dataset Pre, epochs were extracted from the intervals between two rTMS pulse trains. Only epochs of 2.5 s or longer were considered, and the longest epoch was 186.0 s long. The mean epoch length (interval between two rTMS pulse trains) was 15.6 s at a standard deviation of 75.3 s. For dataset Post, epochs were extracted from a time window [0, 2.5]s relative to the last (i.e., 40^{th}) pulse of each pulse train. A band-pass filter (FIR, 6-13 Hz, order 63) was applied bi-directionally to attenuate oscillatory signal components at frequencies outside the alpha band.

Inter-trial phase coherence (ITPC) is commonly used for quantifying event-related phase modulation. ITPC is a scalar value that ranges from [0, 1] and is derived from an ensemble of phase values at a particular time point in trials. A value closer to 0 indicate slow phase alignment among the trials at that particular time point, while an ITPC value closer to 1 indicates high alignment of phase angles across trials at that point. As a simple example, if there is a systematic effect across N trials where at time point tₑₓₐₘₚₗₑ oscillatory activity shows similar phase (e.g., close to “peak” of a sine wave), it was expected for the single ITPC value derived at time point tₑₓₐₘₚₗₑ for these N trials to be closer to 1 rather than 0. In order to identify effects most relevant to the rTMS treatment, an analysis on electrodes at (F3) and adjacent to (FP1, F7) the stimulation site over DLPFC (the same channels were previously used to determine IAF) was conducted.

The accuracy of the phase estimation of the Hilbert transform for each pulse train from each session is dependent on the signal to noise ratio (SNR) of each pulse train (the ratio of the quasi-alpha (6-13 Hz) wave to other EEG components (1-30 Hz)). This approximation based on fast Fourier transform (FFT) has errors in the energy sense due to the fact that Hilbert transformation is a unitary operator in the L2 space, so instead of averaging across trials for the phase coherence calculation, each trial was first weighted by its power in the inter pulse train period (epoched dataset Pre; see FIG. 4).

Relative power was used to calculate the trial weight of phase for each pulse interval with the consideration of consistency and comparability within one session. Relative power was defined as the ratio of absolute quasi-alpha power to the total power calculated from 1 to 30 Hz (spanning delta, theta, alpha and beta bands, see eq (5) below). Quasi-alpha power was calculated as the integrated power between 6 and 13 Hz which is the range used to identify the IAF for each subject during the rTMS triggering. The power of the entire spectrum (1-30 Hz) was calculated by Welch's power spectral density (PSD) estimation method, for which the complete epoch was segmented into eight windows that overlapped 50%. The approximate integrals of absolute quasi-alphapower (6-13 Hz) and total frequency band (1-30 Hz) were calculated with the trapezoidal method of non-unit but uniform spacing which is determined by the frequency resolution (frequency resolution was 0.2441 Hz). More formally, trial weight was calculated as follows:

$${α}_{n , S} = {\int }_{6}^{13} {P}_{n , S , f}^{targeted} df = {\int }_{6}^{13} \frac{1}{3} ( {P}_{n , S , f}^{FP 1} + {P}_{n , S , f}^{F 3} + {P}_{n , S , f}^{F 7} ) df \qquad ( 4 )$$

$${α}_{n , S} = \frac{{α}_{n , S}}{{\int }_{1}^{30} {P}_{n , S , f}^{targeted} df} \qquad ( 5 )$$

$${ω}_{n , S} = \frac{{α}_{n , S}}{{\sum }_{n = 1}^{n = 75} {α}_{n , s}} \qquad ( 6 )$$

Where α_{n,S} is the absolute qusi-alpha power for trial n form session S; ∫_{f1}^{f2}p_{n,S,f}^{j}df is the integral of power between frequency f1 and f2 of channel j for trial n from session S,j={FP1, F3,F7}; targeted refers to the near targeted area which includes FP1, F3, and F7; α_{n,S} is the relative power for trial n from session S; ω_{n,S} is the trail weight for trial n for session S.

After the trial weight calculation, the Hilbert Transform(H{⋅}) was applied to the dataset Post (see FIG. 4) to estimate the instantaneous phase 4n,j(t) of signal xn,j(t) locked to the last TMS pulse for trial n and channel j, where t∈[0, 2.5](s), φ(t)∈[−π, π].

$$φ ( t ) = {tan}^{- 1} ( \frac{H \{ x ( t ) \}}{x ( t )} ) , φ ( t ) ∈ [ - π , π ] \qquad ( 7 )$$

The example then transformed the phase angle back to the analytic signal Z_{n,j}(t) in the real and complex domain using Euler's form

Zₙⱼ(t)=re^{iφ}^{ₙⱼ}^{(t)}=r cos(φ_{n,j}(t))+i×r sin(φ_{n,j}(t)),r=1 (8).

Instead of simply averaging Z_{n,j}(t) across the trials (i.e., subscript n), the example calculated a weighted average, where the analytic signal for each trial was weighted by coefficients ω_{n,S} that were derived based on relative quasi-alpha power for that trial, as described earlier (see Equation (6)). That way the absolute part of the intermediate result, Z_{j,S}(t), represented trial weighted ITPC for channel (electrode) j, which resulted in a 3*625 matrix of ITPC values for each session. Each row represents one channel (FP1, F3, and F7) and columns represent the samples in a trial (width of epoch of dataset Post, 2.5 s*250 Hz sampling rate). Finally, for the spatial average, the example calculated the circular mean across these three EEG channels and obtain the absolute value, which is the post-stimulation ITPC_{S}(t) of the near target region. Based on these resulting time series, it was determined the ITPC for the time range [0, 2.5]s post rTMS pulse train (see FIG. 5).

$${\overline{Z}}_{j , S} ( t ) = {\sum }_{n = 1}^{n = 75} {e}^{i \times {φ}_{n , S , j} ( t )} \times {ω}_{n , S} \qquad ( 9 )$$

$${ITPC}_{S} ( t ) = \vert \frac{1}{3} {\sum }_{j = 1}^{j = 3} {\overline{Z}}_{j , S} ( t ) \vert = \vert \frac{1}{3} {\sum }_{j = 1}^{j = 3} {\sum }_{n = 1}^{n = 75} {e}^{i \times {φ}_{n , S , j} ( t )} \times {ω}_{n , S} \vert \qquad ( 10 )$$

Where ITPC_{S}(t) refers to the average IPTC value for seeion S at time t post rTMS; |Z_{j,S}| refers to the ITPC value of channel j from session S; φ_{n,S,j}(t) is the instantaneous phase of channel j from trial n of session S; ω_{n,S} is the trail weight for trial n of seeion S.

At the subject level, in order to see how this brain synchronization after a rTMS pulse train changes across sessions, Spearman correlation (Spearman's ρ) was used to capture the relationship between the first post-stimulation ITPC peak (referred to as ITPC^{max[1]}, which is defined as the first local maximum of the ITPC following the last TMS pulse in a train, see FIG. 5. The range of Spearman's ρ is [−1, 1], with 1 indicating perfect correlation, −1 perfect anticorrelation and 0 that there is no monotonic association between two variables. Illustrated in FIG. 5, processing flow is indicated by the large black arrow which starts at the upper left and goes counterclockwise to the upper right. First, two datasets were generated, one Pre and one Post with respect to the TMS pulse train. The Pre data was used for the trial weight calculation and the Post segment was used for the post-stimulation phase calculation. For each pulse train, the trial weight, ω_{n,S}, was calculated based on relative alpha power of the Pre segment. The phase of the Post segment was obtained by a Hilbert transform, shown in polar coordinates by applying Euler's formula. This process was repeated for each pulsetrain of one session, and the results of each pulse train were combined via Equation (9) resulting in the trial weighted-phases, shown as polar coordinates, from t=0 s to t=2.5 s post rTMS pulse train. In FIG. 5, trials with greater weight are shown with darker blue, while a smaller trial weight is shown as lighter blue. Using Equation (10), the trial weighted ITPC was calculated for each electrode in a region (shown here is the target region including electrodes FP1, F3, and F7) and the mean of the ITPC was also calculated across the three electrodes. The magnitude of vectors (mean, black) were plotted in the time window t=[0, 2]s and the first peak of ITPC (ITPC^{max[1]}) was taken to present the post-stimulation ITPC value for that session. Finally, it has been analyzed how this time series of ITPC^{max[1]} changes across sessions for each subject # P, as shown in the upper right corner which uses a SYNC subject who has increasing phase entrainment as an example. The example used a generalized linear mixed-effects model (GLMM) to analyze changes in ITPC^{max[1]} across sessions as a function of treatment arm (SYNC vs UNSYNC). A GLMM is an extension to the generalized linear model (GLM) in which the linear predictor contains random effects in addition to the usual fixed effects. The general form of a GLMM is as follows:

y=Xβ+Zμ+ϵ (11)

Where y is the outcome variable; X represents the predictor variables; β is a column vector of the fixed-effects regression coefficients; Z is the design matrix for the random effects (the random complement to the fixed X); μ is a vector of the random effects (the random complement to the fixed β); and ϵ is a column vector of the residuals.

The example used the GLMM in Matlab (Statistics and Machine Learning Toolbox, Matlab 2018b, Mathworks, USA) to investigate the relationship between ITPC^{max[1]} and the corresponding independent variables which include stimulation frequency (IAF), relative quasi-alpha power, α_{P} session number of each treatment, and subject's treatment group (SYNC or UNSYNC). The fixed-effects in the model included stimulation frequency, relative quasi-alpha power, treatment group, session number, the interaction between treatment group and relative power, and the interaction between treatment group and session number. The subject difference was modeled by grouping variable sub as random-effects. Therefore, the final model is:

$$ln ( \frac{{ITPC}^{max [ 1 ]}}{1 - {ITPC}^{max [ 1 ]}} ) ∼ stimf + ( session + {\overline{α}}_{P} ) * condition + ( 1 \vert sub ) + ϵ , ln ( \frac{{ITPC}^{max [ 1 ]}}{1 - {ITPC}^{max [ 1 ]}} ) ∈ [ 0 , 1 ]$$

Where ITPC^{max[1]} refers to the first post-stimulation ITPC peak value for each session; stimf refers to the stimulation frequency for each session; session is the corresponding session number (e.g., the first treatment is 1); α_{P} is the relative quasi-alpha power for each session; condition is the SYNC(1) or UNSYNC(−1) group; sub represent each subject (e.g., the first subject is 1). In addition, because the range of ITPC^{max[1]} is between 0 and 1 (ITPCϵ[0, 1], also ITPC^{max[1]}ϵ[0, 1]), the logit link function is applied in this linear model.

Fifteen patients with treatment resistant depression were enrolled and assigned randomly to either of the two treatment arms, SYNC (experimental treatment) or UNSYNC (active comparator) (see Table 1). A target phase of quasi-alpha EEG, defined as the phase at which a TMS pulse to left DLPFC evoked strongest activity in dorsal anterior cingulate cortex (dACC), was determined for every subject in a single session of combined fET (in Example 2). Patients participated in 30 treatment sessions, only one session per work day for six weeks (extended to seven weeks if sessions were skipped). For these treatment sessions, participants were seated comfortably in an adjustable armchair with the EEG-rTMS setup (see FIG. 1A-1B). Every closed-loop EEG-rTMS treatment session (see FIG. 4) started with 5 min of resting state recording where an individual alpha frequency (IAF) and triggering threshold (RMSE) was determined. The closed-loop EEG-rTMS treatment for one session lasted approximately 30 min, and patients received 75 pulse trains of rTMS, with 40 pulses per train over the left DLPFC at 120% of intensity relative to their individual motor threshold. The interval between pulses in a pulse train was set to Δtᵢₚᵢ=1/IAF (e.g., 125 ms for a patient with alpha frequency of 8 Hz) for both the SYNC and UNSYNC groups. For patients who were assigned to the group SYNC, the first TMS pulse in each train of 40 pulses was triggered at the individual's preferred phase, as determined from the initial fET session (φ_{targ}=φₚᵣₑ). For patients in the UNSYNC group, the preferred phase was not targeted, but instead the target phase was drawn randomly from a uniform distribution over the range [0, 2π] for the first pulse in every rTMS pulse train (φ_{targ}˜U(0, 2π)).

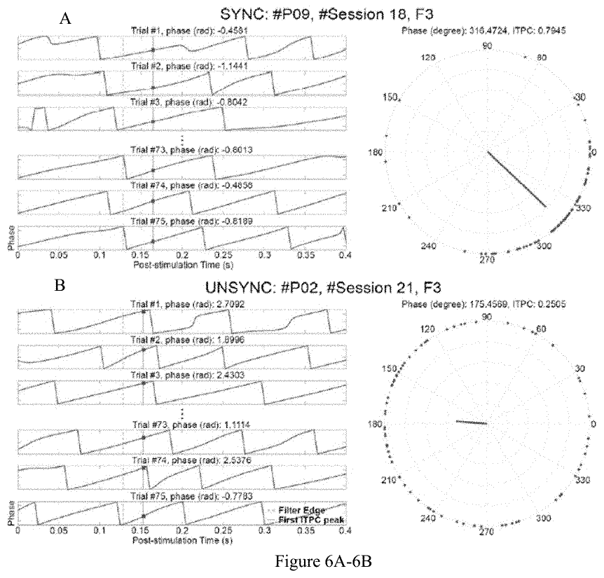

FIG. 6A-6B shows an illustration of how the ITPC, for a given session, is estimated from the raw data for both SYNC (FIG. 6A) and UNSYNC (FIG. 6B) subjects. As shown in FIG. 6A-6B, estimate of phase entrainment in the quasi-alpha band (6-13 Hz) at the target electrode (F3) is identified. One session from a SYNC subject (# P09, # Session 18) and one session from an UNSYNC subject (# P02, # Session 21) are presented. For each trial of a session, the phase of the Post stimulation segment was obtained via Hilbert transform after alpha-band filtering, with the phase value shown (line) in each subpanel on the left t=0 refers to the end of one rTMS pulse train and the red dashed line in each subpanel indicates the filter edge (t=0.128 s). The black solid line is the corresponding time point where the first post-stimulation ITPC peak ITPC^{max[1]} was detected (one value was calculated per session). The intersection (dot) of the blue line and black line is the corresponding phase value of ITPC^{max[1]}. These points, across all trials of a session, are combined via Equation (8) resulting in the phase points shown as the green dots on the polar coordinates (r=1) on the right. Using Equations (9) and (10), the trial weighted ITPC^{max[1]} (bar) is calculated for electrode F3 based on these points. In this example, the value of ITPC^{max[1]} for this SYNC subject is two times greater (ITPC^{max[1]}=0.7945) than this UNSYNC subject (ITPC^{max[1]}=0.2505) indicating much greater entrainment on F3.

Post-stimulation, there is an increase across sessions in the first ITPC peak (or ITPC^{max[1]}) around the stimulation site (left DLPFC; based on electrodes F3, FP1 and F7) for SYNC patients relative to the control group UNSYNC (Spearman's rank correlation coefficient, Table 3). Specifically, for the SYNC experimental group, three of seven subjects showed a statistically significant (p<0.05) increase in the post-stimulation ITPC^{max[1]} over sessions (see Table 3), suggesting that more days of treatment with phase synchronized rTMS was associated with increasingly greater post-stimulation alignment in quasi-alpha phase between trials. For the UNSYNC control group, this effect was observed for only one of eight subjects (see Table 3).

Table 2 illustrates the number of patients in different phase change direction for each group.

TABLE 2

| Phase Difference | SYNC | UNSYNC |
|---|---|---|
| Closer | 5 | 1 |
| Farther | 2 | 5 |

TABLE 3

| Subject | Condition | P | p-value |
|---|---|---|---|
| P01 | unsync | 0.4612 | 0.0110(*) |
| P02 | unsync | 0.1462 | 0.4392 |
| P03 | unsync | 0.0670 | 0.7244 |
| P04 | unsync | 0.0056 | 0.9775 |
| P05 | unsync | −0.0478 | 0.8015 |
| P06 | unsync | −0.0216 | 0.9102 |
| P07 | unsync | −0.0170 | 0.9323 |
| P08 | unsync | −0.2796 | 0.1343 |
| P09 | sync | 0.7320 | 0.0000(***) |
| P10 | sync | 0.4585 | 0.0115(*) |
| P11 | sync | 0.4011 | 0.0391(.) |
| P12 | sync | 0.3112 | 0.0944(.) |
| P13 | sync | 0.1773 | 0.3471 |
| P14 | sync | 0.0553 | 0.7795 |
| P15 | sync | −0.1430 | 0.4492 |

Spearman correlation between post-stimulation trial weighted first poststimulation ITPC peak (ITPC^{max[1]})and Session; (***) indicates significant under a 99.9% confidence level; (**) indicates significant under a 99% confidence level; (*) indicates significant under a 95% confidence level; (.) indicates significant under a 90% confidence level.

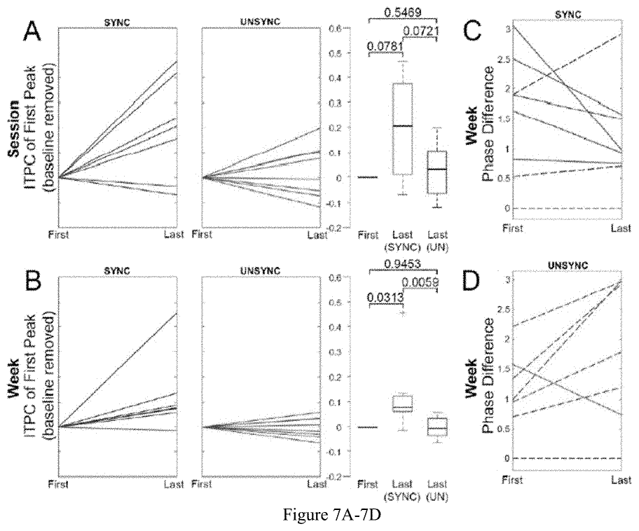

FIG. 7A-7D compare the changes in the post-stimulation ITPC^{max[1]} for SYNC and UNSYNC groups both by session and by week. It shows that five SYNC group subjects show an increase in quasi-alpha entrainment represented by positive ΔITPC^{max[1]} between the first and last session (where the first session value was subtracted as baseline, see FIG. 7A). Group level effects were tested with non-parametric tests. A two-sided Wilcoxon signed rank test was used to test the difference between the first and last session within each group, where the null hypothesis was that the difference between the first and last session comes from a distribution with zero median. A Wilcoxon rank sum test was also used to test the difference between the first and last session across groups, with the null hypothesis being they come from the same population. As there can be noise/variation in the measurement of each single treatment session, a similar analysis by averaging sessions across week was conducted. This analysis was similar to the session comparison, except ΔITPC^{max[1]} was calculated between the first and last week (where all sessions in a week were averaged and the ITPC of the first week was subtracted as baseline, see FIG. 7B. The group level effect is the most significant (p=0.0059) between SYNC and UNSYNC groups in the week comparison. This indicates that though the impact can be variable across individual days, EEG synchronized rTMS treatment is associated with greater post-stimulation quasi-alpha entrainment, compared to unsynchronized treatment, over the long-term across multiple sessions extending over weeks.

The relationship between each subject's peak quasi-alpha entrainment phase (pent) and their individual preferred phase that maximally engaged the ACC target (φₚᵣₑ from pre-treatment scan and φₚₒₛₜ from post-treatment scan) are investigated. Here, pent is the corresponding phase at the time when the first post-stimulation ITPC peak, ITPC^{max[1]}, was found (see FIG. 6A, i.e., the entrainment phase calculated based on electrode F3 for subject t # P09, # Session 18 is 316.4724°). As mentioned earlier, the pre-treatment target phase (φₚᵣₑ) was determined using a simultaneous fET scan. A second post-treatment fET scan at the end of the six-week treatments was performed to determine the target phase at that point (φₚₒₛₜ), since treatment itself could potentially affect the phase relationship between the TMS and the activity at the therapeutic target, namely the ACC. First, the corresponding φₑₙₜ at the time that ITPC^{max[1]} was detected for the treatments of the first and last week, where each week included 5 treatment sessions. Then the circular mean was calculated to represent the entrainment phase of the first (φₑₙₜ,1^{st}) and last week (φₑₙₜ,6^{th}). For the first week, the differences, for each subject, of φₑₙₜ,1^{st} and φₚᵣₑ were computed, while for the last week the differences of φₑₙₜ,6^{th} relative to φₚₒₛₜ were computed. FIG. 7C and FIG. 7D show the results for each treatment group. For the SYNC group, 5 out of 7 subjects' phase differences (entrainment phase minus target phase) decrease from the first to the last week, indicating that the entrainment phase and preferred target phase are converging over the treatment sessions. Conversely, in the UNSYNC group, this convergence happens in only 1 out of 6 subjects. Note that two UNSYNC subjects are excluded here because their post-treatment fET scans were not available.

A Kruskal-Wallis test was used to test the null hypothesis that the phase difference in the first and last week in each group (SYNC vs UNSYNC) comes from the same distribution. Treating the direction of the phase changes (clockwise vs counterclockwise) as different and considering the magnitude of the differences, the null hypothesis (p=0.0455) at the 5% significance level can be rejected. A second test to investigate was performed to determine whether an increase/decrease of phase was different across the groups, regardless of the magnitude of the individual changes for each subject. Fisher's exact test to Table 2 was applied to test if there are nonrandom associations between the categorical findings of increase/decrease of phase difference in SYNC and UNSYNC groups. The result of Fisher's test is p=0.1026, thus unable to reject the null hypothesis of no nonrandom association between the categorical variables (SYNC vs UNSYNC) at the 5% significance level. This finding, together with the analysis taking the magnitude of the phase difference into account and the significant increase in entrainment over time, is consistent with an interpretation that there is a shift in phase that is induced in the SYNC group. Thus the individual entrainment phase appears to move toward the individual target phase, i.e., toward the phase associated with the strongest BOLD activation in the ACC after subjects received rTMS treatment synchronized to their quasi alpha activity (mainly alpha activity).

A significant group level effect was identified, where ITPC^{max[1]} increased across sessions only when rTMS was synchronized to individual preferred phase (SYNC group). Specifically, a statistically significant effect of the interaction between the factors session-number (1-30) and treatment group (SYNC and UNSYNC) on ITPC^{max[1]} as the dependent variable (generalized linear mixed effects model; b=0.0307, p=0.0000, R²=0.4329; see Table 4) was identified.

TABLE 4

| Name | Estimate | SE | t-Sat. | DF | p-value | Lower-CI | Upper-CI |
|---|---|---|---|---|---|---|---|
| (Intercept) | 0.3929 | 0.3371 | −1.1655 | 435 | 0.2445 | −1.0556 | 0.2697 |
| Stimf | 0.0405 | 0.0256 | −1.5803 | 435 | 0.1148 | −0.0909 | 0.0099 |
| \hat{α}_{P} | −0.3128 | 0.5875 | −0.5323 | 435 | 0.5948 | −1.4675 | 0.8420 |
| Session | 0.0013 | 0.0038 | 0.3487 | 435 | 0.7275 | −0.0062 | 0.0089 |
| Condition | −0.1355 | 0.3145 | −0.4309 | 435 | 0.6668 | −0.7537 | 0.4827 |
| \check{α}_{P}:condition | −1.5174 | 0.8257 | −1.8378 | 435 | 0.0668 | −3.141 | 0.1054 |
| session:condition | 0.0307 | 0.0058 | 5.2647 | 435 | 0.0000 | 0.0192 | 0.0422 |

Fixed effects coefficients (95% CIs).

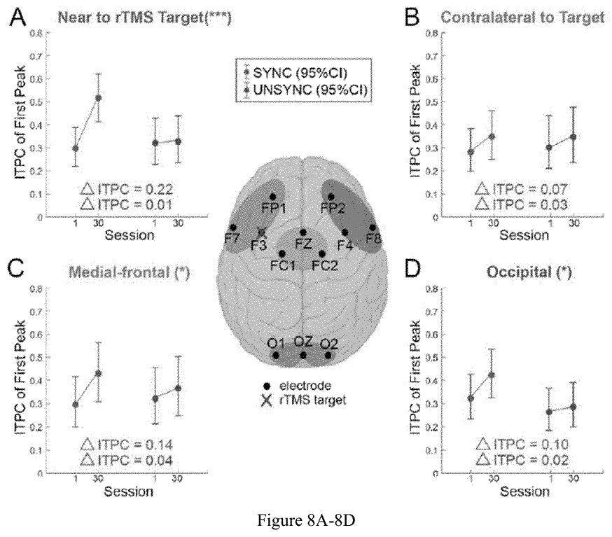

FIG. 8A-8D is an illustration for following the significant effect observed for the interaction between session and group (see Table 4), It has shown the effect of treatment session separately for UNSYNC and SYNC groups (i.e., marginal effect) across four different regions of interest (ROIs) based on the GLMM prediction. The central figure defines the ROIs and the electrodes used in the analysis. All ROIs consist of three electrodes. rTMS is applied over the left DLPFC (over electrode F3) for all subjects. FIG. 8A shows the model prediction of changes in ITPC^{max[1]} between the first and last session for SYNC and UNSYNC groups at the ROI near the rTMS target ROI, FIG. 8B shows contralateral to the target ROI, FIG. 8C shows the model in the medial-frontal ROI and FIG. 8D shows model in the occipital ROI. The interaction-term of session and SYNC/UNSYNC group in the GLMM was highly significant in FIG. 8A (***, p<0.01), significant in FIG. 8C and FIG. 8D (*, 0.01<p<0.05) but not significant in FIG. 8B.

FIG. 8A shows the marginal effect of session-number on ITPC^{max[1]} for the SYNC group on the near target region which includes electrodes FP1, F7 and F3 (ITPC₁ₛₜ^{max[1]}=0.2980, ITPC₃₀ₜₕ^{max}=0.5182, ΔITPC^{max[1]}=0.2202), see FIG. 7A). No significant effect was observed for an increasing session-number on ITPC^{max[1]} for the UNSYNC group ITPC₁ₛₜ^{max[1]}=0.3204, ITPC₃₀ₜₕ^{max[1]}=0.3289, ΔITPC^{max[1]}=0.0085; see FIG. 8A). No significant effects were found for stimulation frequency (IAF) or session-number and treatment group alone. Random effects covariance parameters are shown in Table 5 (Random effects).

TABLE 5

| | | | Type | Estimate |
|---|---|---|---|---|
| Grou:sub | (Intercept) | Intercept | std | 0.33875 |
| (15 Levels) | | | | |
| Group:Error | Sqrt(Dispersion) | — | — | 0.10561 |

The same analysis was conducted as a function of the EEG channels used to compute the post-stimulation ITPC^{max[1]} (e.g., contralateral to rTMS target, see FIG. 8B). The ITPC^{max[1]} increase across sessions (Δ ITPC_{max[1]}) is largest near the rTMS targeted area and fades to be nonsignificant in the area contralateral to the rTMS target (see FIG. 8A-8D).

In the disclosed embodiments, differences in the consistency of TMS phase-locked responses were evaluated using an ITPC comparison between patients in SYNC versus UNSYNC groups. It has shown that ITPC^{max[1]} observed after TMS pulse trains over the left DLPFC region significantly increased across treatment sessions for patients who received SYNC rTMS treatment, while it did not for patients in the active control condition UNSYNC. This result suggests that long term continuous synchronized rTMS treatments over left DLPFC could lead to greater brain synchronization and entrainment in the targeted area in treatment-refractory MDD patients.

Despite rTMS being approved as a treatment for MDD, there continues to be a need to improve its efficacy. As evidenced by the fET system in certain embodiment, synchronizing the TMS pulse to an individual's brain state over long periods of time is a method that is important for reaching deep areas such as the ACC.

For patients that received SYNC condition treatment (i.e., onset of rTMS time-locked to preferred instead of random phase), the consistency of the TMS phase-locked response across trials increased as the number of treatment sessions increased. There was an increase in the first ITPC peak value post-stimulation, ITPC^{max[1]}, across sessions. For patients in the UNSYNC group, no such effect was established. On subject-level, one participant in the UNSYNC group showed statistically significant phase entrainment at a considerable correlation strength. In conclusion, the efficacy of rTMS has been improved with synchronized rTMS pulse triggering, by showing an increase in brain synchronization across treatments. Furthermore, fET or EEG-rTMS has been proved to be advantageous to improving the physiological and therapeutic effects of phase-synchronized stimulation in patients with MDD. Specifically, when rTMS is applied over the DLPFC and synchronized to the patient's prefrontal quasi-alpha rhythm, patients develop strong phase entrainment over a period of weeks, both over the stimulation site as well as in a subset of areas distal to the stimulation site. In addition, at the end of the course of treatment (e.g., multiple sessions treatment), this group's entrainment phase shifts to be closer to the phase that optimally engages the distal target, namely ACC. These entrainment effects are not observed in the group that is given rTMS without initial EEG synchronization of each TMS train.

The description herein merely illustrates the principles of the disclosed subject matter. Various modifications and alterations to the described embodiments will be apparent to those skilled in the art in view of the teachings herein. Further, it should be noted that the language used herein has been selected for readability rather than to delineate or limit the disclosed subject matter. Accordingly, the disclosure herein is intended to be illustrative, but not limiting, and can be implemented in various configurations of hardware and/or software, and are not intended to be limited in any way to the specific embodiments presented herein.

What is claimed is:

1. A system for identifying an alpha phase in brain of a subject, comprising:
a processor configured to:
process one or more of functional magnetic resonance imaging (fMRI) data and electroencephalogram (EEG) data, wherein the fMRI data and EEG data are simultaneously acquired;
trigger a transcranial magnetic stimulation (TMS) pulse;
analyze the fMRI data if any, the EEG data if any and the TMS pulse; and
determine the alpha phase,
wherein the alpha phase evokes strong activity in dorsal anterior cingulate cortex (dACC).

2. The system of claim 1, wherein the processor further is configured to determine a target alpha phase associated with the strong response in the dACC as measured by blood oxygen level dependent (BOLD) signal.

3. The system of claim 1, wherein the EEG data is acquired using a MR-compatible EEG system.

4. The system of claim 1, wherein a level of activation of the anterior cingulate cortex (ACC) varies with the TMS pulse applied to dorsal lateral prefrontal cortex (DLPFC).

5. The system of claim 5, wherein the DLPFC is depended on timing of the applied TMS pulses relative to the phase of an EEG alpha rhythm of the subject.

6. The system of claim 1, wherein the alpha phase is determined in the EEG data at the time of each TMS pulse.

7. The system of claim 1, wherein the TMS pulse is synchronized to the patient's prefrontal quasi-alpha rhythm.

8. A closed-loop electroencephalogram (EEG)-repetitive transcranial magnetic stimulation (rTMS) system, comprising:
a processor configured to
acquire EEG data;
trigger a rTMS pulse;
process the EEG data and the rTMS pulse; and
determine a target alpha phase,
wherein a first alpha phase is determined before the target alpha phase is determined.

9. The system of claim 8, wherein rTMS is synchronized to the EEG alpha phase.

10. The system of claim 8, wherein the first alpha phase is determined by functional magnetic resonance imaging (fMRI) data, electroencephalogram (EEG) data, and a trigger transcranial magnetic stimulation (TMS) pulse.

11. A method of determining a target alpha phase in brain of a subject, comprising:
performing a first simultaneous scan to determine a first alpha phase which evoked strongest activity in dorsal cingulate cortex (dACC);
performing a treatment of multiple sessions to the subject; and
performing a second simultaneous scan at the end of the subject's treatment to determine a target alpha phase after the treatment was complete;
wherein the simultaneous scan uses functional magnetic resonance imaging (fMRI) data, electroencephalogram (EEG) data, and a trigger transcranial magnetic stimulation (TMS) pulse.

12. The method of claim 11, wherein a simultaneous EEG is recorded.

13. The method of claim 11, wherein a motor threshold is measured on the subject's left motor cortex.

14. The method of claim 13, further comprising adjusting a TMS output voltage until an involuntary thumb twitch is observed in the patient before the scan.

15. A method for treating depressive disorder, comprising delivering EEG-triggered repetitive transcranial magnetic stimulation (rTMS) to a subject, wherein the rTMS is synchronized to the subject's prefrontal EEG quasi-alpha rhythm.

16. The method of claim 16, the rTMS is applied on the dorsal prefrontal cortex (DLPFC).
